(19) United States
(12) Patent Application Publication
(10) Pub. No.: US 2007/0197953 A1
(43) Pub. Date: Aug. 23, 2007

(54) Magnetic particles for therapeutic treatment

(76) Inventors: Robert Andrew Slade, Oxon (GB); Michael Eaton, Berkshire (GB)

Correspondence Address: THELEN REID BROWN RAYSMAN & STEINER LLP, P. O. BOX 640640, SAN JOSE, CA, 95164-0640, US

(21) Appl. No.: 10/565,533

(22) PCT Filed: Jul. 16, 2004
(86) PCT No.: PCT/GB04/03117

§ 371 Date: Dec. 13, 2006

(30) Foreign Application Priority Data
Jul. 18, 2003 (GB) 0316912.5

(51) Int. Cl. A61N 1/30

(52) U.S. Cl. 604/19

(57) ABSTRACT

Magnetic particles are used in therapeutic applications to disrupt a biological material. The particles have intrinsic magnetization, said magnetization being stabilised by inherent magneto-crystal line anisotropy and/or by shape anisotropy. By applying a magnetic field, the magnetic particles are induced to rotate when localised at the target biological material, thereby disrupting the material.

### FIELD OF THE INVENTION

This invention relates to the treatment of disorders associated with the accumulation of a biological material or an aberrant cellular or tissue structure. More particularly, this invention relates to the treatment of such disorders by mechanical disruption.

### BACKGROUND TO THE INVENTION

In recent years, there have been many advances in the treatment of disorders associated with aberrant cellular or tissue structures, by the selective targeting of the structures with appropriate therapeutic agents. In particular, in the context of tumour therapy, there have been significant advances in technologies that permit the localisation of appropriate chemotherapies at the tumour site. However, chemotherapy is often compromised by adverse systemic toxicity, which limits the dose of drug that can be administered, or is limited by the appearance of multi-drug resistance. One particular difficulty results from the need to control the cytotoxicity until after localisation at the tumour site, to prevent non-specific cell damage (Eaton, Immunoconjugates: Current Status and Future Potential, Journal of Drug Targeting, 2002; 10(7):525-527).

More recently, there have been investigations into the use of magnetic particles to treat tumours via a process referred to as “hyperthermia”. This process relies on localising magnetic particles at a tumour site and applying a magnetic field from a high frequency AC magnet, to induce magnetic hysteresis loss. The heat dissipating from the particles damages the tumour cells resulting in cell death. A description of this process is found in Jones et al., Int. J. Hyperthermia, 2002; 18(2): 117-128 and also in Jordan et al., Journal of Magnetism and Magnetic Materials, 2001; 225:118-126.

The benefit of this proposed therapy is that the magnetic particles can be administered to a patient in a relatively inert form, and the destructive ability of the particles can be induced selectively, when the particles are localised at the target site. This avoids the uncontrolled cytotoxicity that may be experienced using chemotherapeutic agents. However, a particular disadvantage of the hyperthermia method is that a high concentration of particles must be accumulated in the tissue to be treated to cause a sufficient temperature rise for cell lysis. The heating is macroscopic (ie: not localised to the target cells) with the result that tissue necrosis can occur.

U.S. Pat. No. 6,514,481 describes the targeting of spherical magnetic nanoparticles less than 100 nm in diameter, to a cellular location, with subsequent application of a DC magnetic field, to destroy the targeted cells. The nanoparticles are prepared from iron oxide, e.g. Fe₂O₃, and an applied magnetic field of 7 Tesla is shown to be required to achieve in vitro cell death.

While the results achieved using this process are of interest, the requirement for a very strong magnetic field limits the suitability of the process for clinical applications due to the high cost of whole-body hardware for generating fields above about 2 Tesla, and the danger of damaging healthy tissue due to causing motion in non-localised naturally occurring or contaminant particles.

WO-A-01/17611 discloses the use of nanoparticles in a hyperthermia process that also requires the induction of shearing forces. The shearing forces are induced by applying an alternating magnetic gradient field. The only magnetic particles disclosed are metal oxides. The magnetic gradient field causes the particles to experience a translation force acting to move them along the field gradient; alternating the gradient field moves the particles in opposite directions, thereby inducing a “vibration” effect.

Halbreich et al., Journal of Magnetism and Magnetic Materials, 2002; 248: 276-285 describes a process referred to as “magnetocytolysis”. The particles used in the process are stated to be made of magnetite (iron oxide) and an optimum field oscillation frequency is indicated to be 1000 KHz. The mechanism of magnetocytolysis is not specified but is indicated to be related to locally generated intense gradient fields at the boundaries between regions with differing concentrations of magnetic nanoparticles, such as cell membranes.

U.S. Pat. No. 6,231,496 discloses the use of nanoparticles having a sharp end, which are to be embedded within the lining of the uterus by the application of a magnetic field. Microwave radiation is then applied to generate tissue heating resulting in the destruction of the uterine lining, to achieve sterilisation.

U.S. Pat. No. 5,067,952 discloses the use of ferromagnetic particles for use in treating tumours by hyperthermia. The nanoparticles may also be caused to vibrate by the use of ultrasonic oscillation. Hyperthermia is carried out by applying an electromagnetic field at a frequency of 13.65 MHz. The destruction of the tumour is therefore a heat-based mechanism.

U.S. Pat. No. 5,236,410 discloses the use of hexaferrite particles in the treatment of tumours, via hyperthermia. The particles are said to be from 500 nm to 7 μm in size. The magnetic field frequency required to induce hyperthermia is approximately 500 MHz.

Although the above references show that magnetic particles have found use in therapeutic applications, there is still a requirement for improved processes for inducing cell death using magnetic particles, which do not rely on high field strengths and/or high frequency variation of the magnetic field, nor on procedures which induce localised or general temperature rise in tissue.

### SUMMARY OF THE INVENTION

The present invention is based, at least in part, on the understanding that effective destruction of materials, including biological/cellular structures can be achieved by physical disruption, by exposing selected magnetic particles that have been localised at the target site to a relatively weak and slowly varying magnetic field, thereby causing them to rotate and align with the applied field. The cost of hardware required to generate such fields at whole body scale is thereby substantially reduced.

According to a first aspect of the invention, magnetic particles are used in the manufacture of a medicament for administration to a patient to treat a disorder associated with a cellular or tissue structure, or the accumulation of an undesirable biological material, wherein the or each particle is preferably adapted to localise at or within the structure or material, and wherein the treatment is intended to be carried out by applying a magnetic field, to induce the or each particle to rotate, to thereby disrupt the structure or material, wherein the or each magnetic particle has intrinsic magnetization, said magnetization being stabilised by inherent magneto-crystalline anisotropy and/or by shape anisotropy.

According to a second aspect of the invention, a method for disrupting a material, comprises the steps of:
(i) localising one or more magnetic particles at or within the material; and
(ii) applying a magnetic field to the or each magnetic particle, to induce particle rotation and thereby disrupt the material, wherein the or each magnetic particle has intrinsic magnetization, said magnetization being stabilised by inherent magneto-crystalline anisotropy and/or by shape anisotropy and wherein the applied magnetic field direction or amplitude with respect to the material is varied over time.

Contrary to the processes of the prior art that rely on hyperthermia to achieve cell death, the present invention relies on inducing rotation of the particles with sufficient torque to achieve a mechanical disruption of the cell structure or other biological material. With the correct particle design, particle rotation can be achieved using a magnetic field strength significantly lower than that disclosed in U.S. Pat. No. 6,514,481, and with a much lower field oscillation frequency (ie: lower field slew rate) than that disclosed by Halbreich at al, thereby permitting clinical development of the therapy.

According to a third aspect of the present invention, apparatus for disrupting a material, including a cellular or tissue structure, comprises a magnetic field generator for generating a magnetic field in a working volume; one or more magnetic particles localised at or in the material in the working volume, the or each magnetic particle having intrinsic magnetization, said magnetization being stabilised by inherent magneto-crystalline anisotropy and/or by shape anisotropy; and a control system for causing a change in the magnetic field in the working volume with respect to the material so as to rotate the magnetic particle.

According to a fourth aspect of the invention, a magnetic particle having intrinsic magnetization, said magnetization being stabilised by inherent magneto-crystalline anisotropy and/or by shape anisotropy, comprises a targeting moiety bound thereto.

According to a fifth aspect of the invention, a composition comprises a plurality of magnetic particles having intrinsic magnetization, said magnetization being stabilised by inherent magneto-crystalline anisotropy and/or shape anisotropy, the particles further comprising a targeting moiety bound thereto, and the composition further comprising a pharmaceutically acceptable buffer, diluent or excipient.

### DESCRIPTION OF THE DRAWINGS

The invention is described with reference to the accompanying drawings, wherein:

FIGS. 1a to 1c illustrate the differences in magnetisation M of “hard” and “soft” magnetic nano-particles, relative to an applied magnetic field H;

FIG. 2 illustrates the dependence of coercivity on particle size;

FIGS. 3a and 3b illustrate the oblate and prolate spheroid configurations preferred for “soft” magnetic particles; and,

FIGS. 4-7 are schematic diagrams of different types of magnetic field generating apparatus.

### DESCRIPTION OF THE INVENTION

The particles for use in the present invention may be of any suitable size, but will preferably be on the nano scale, i.e. less than 1 μm. The particles may therefore be referred to as nanoparticles. Preferably the particles comprise a magnetic core within a bio-compatible coating, the particle core being from 50 nm to 500 nm in size, more preferably approximately 100 to 150 nm in size. The optimum size will depend, at least in part, on the choice of material. Further reference to the size of the particles should be taken as referring to the magnetic core material and not the total size with any coating, unless there is a statement to the contrary.

In order to be efficacious, a sufficiently large force must be generated to damage critical elements of the cell's structure. To a large extent this depends on the mechanical properties of the cell (in turn described by complex properties of cytoskeletal viscoelasticity, rheology, tensegrity, etc). However, current research suggests that a force of around 100 pN is sufficient to break microtubules (a key cellular structure associated with intracellular transport). For the purposes of describing the invention the embodiments given in this application therefore assume a damage threshold force of 100 pN and describe apparatus capable of generating this threshold force. It will be apparent that the invention is in no way intended to be limited to a threshold force of either above or below 100 pN.

To generate the damage threshold force efficiently requires careful consideration of the practical limitations on both the design of the magnetic core of the nanoparticle, and the whole body therapy magnet. The design parameters for the magnetic core are:

size

shape

material

and for the magnet the primary design parameters are:

size of treatment region (ie: whole body or localised)

magnetic field strength in the treatment region

time dependence of magnetic field strength within the treatment region

The preferred time dependence of the applied magnetic field depends strongly on the viscoelastic properties of the cell. The optimum time dependence must be evaluated by experimentation, but must also fall within practical constraints of magnet hardware, which will be discussed in due course.

For the nanoparticle to be internalised within the cell, it must be of an appropriate size. The preferred maximum dimension of the particle including biocompatible coating is therefore about 200 nm.

Particles having a magnetic moment m will experience torque and translation forces in an applied magnetic field of flux density B. The torque acts to align the magnetic moment with the applied field direction and is given by
Γ=m×B
The translation force, which acts to move the magnetic moment along the applied field gradient towards the region of highest flux density, is given by
F=−grad(m·B)

The variation of B across the nanoparticle is negligible, so this becomes:
F==−m·grad(B)

The magnetic field must have a very large gradient to generate a substantial translation force. In fact the translation force turns out to be negligible in all practical arrangements and henceforth only torque will be considered.

The peak force couple (F_{c}) on a particle experiencing torque Γ and having dimension d (measured in a direction orthogonal to Γ) occurs when m is orthogonal to B:
F_{c}=|Γ|ₚₑₐₖ/d=|m||B|/d

It is clear that to maximise the torque or force couple we need to maximise B and m. We also want to minimise B to keep down the therapy magnet cost, so we are left with the optimisation problem: “maximise |m|| to meet the damage force threshold with minimum |B|”.

The magnetic moment m is related to the particle's magnetization M by volume integration:
m=∫ᵥMdV
Writing M_{eff} as the effective or net magnetization averaged over the whole particle including self-demagnetizing effects:
|m|=V·M_{eff}
so that the peak force couple is given by
F_{C}=VM_{eff}B/d
from which we can conclude that the magnetic flux density B, the particle's volume V and effective magnetization M_{eff} must be as large as possible.

The magnetic flux density needed to generate the required force couple is therefore:
B=F_{C}d/VM_{eff} (1)

Calculations using equation (1) and data from Table 1 (below) show that the minimum preferred size for the magnetic core using the preferred material (hexaferrite) is about 50 nm. Particles smaller than this require much stronger magnetic fields to induce cell lysis. Rare-earth materials allow the use of smaller particles at practical field strengths (1 T), but if rare-earth materials are to be used, they require a coating of an inert biocompatible material to avoid toxicity problems.

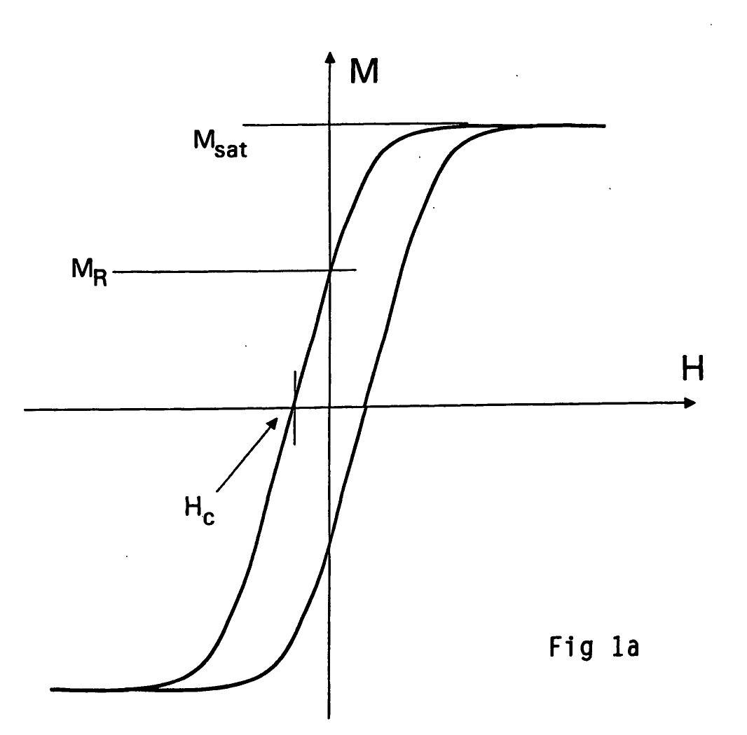

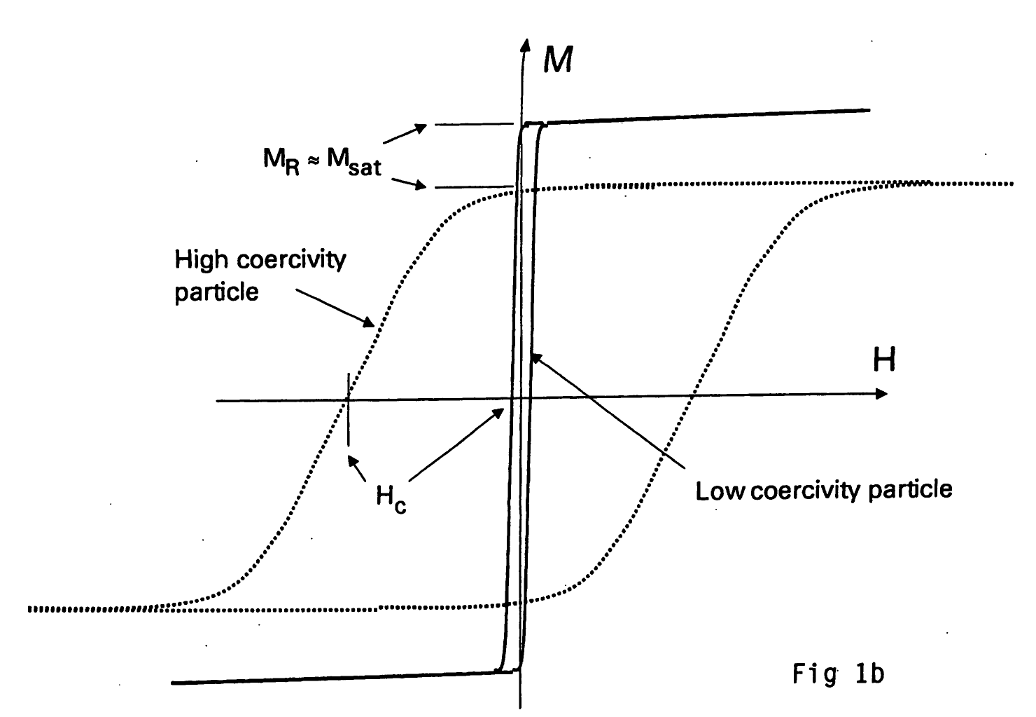

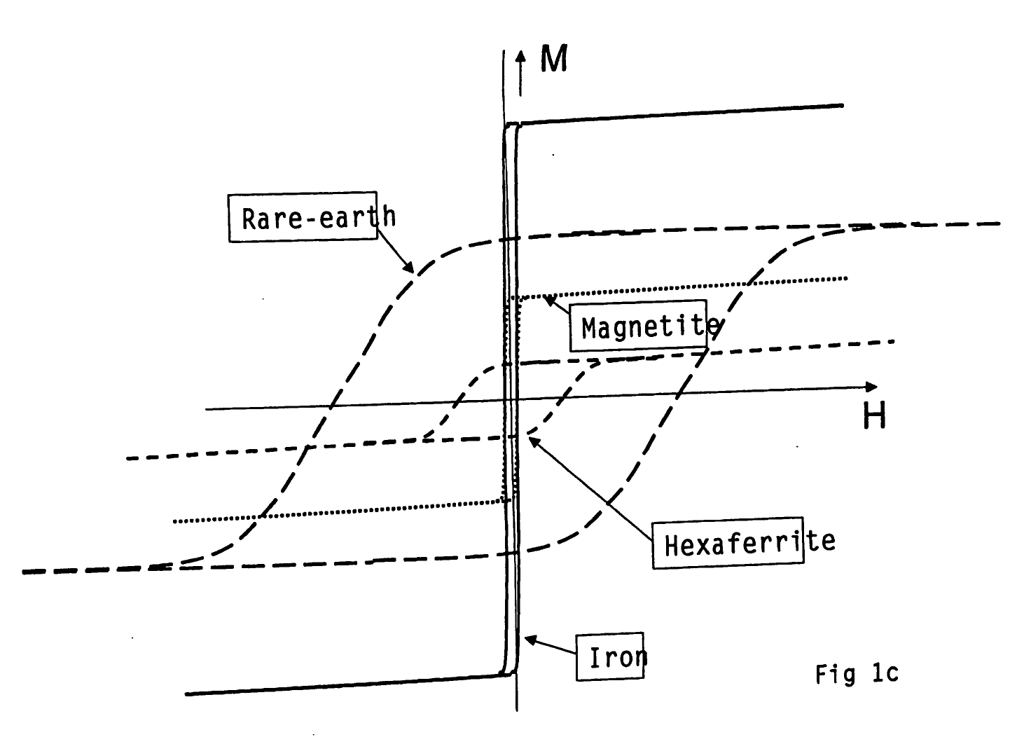

FIG. 1a shows the major magnetization loop of a bulk sample of typical ferromagnetic material, plotting magnetization M against applied field H. A ferro- or ferr-magnetic material is composed of ˜10¹² to 10⁵ domains per cubic centimetre, each domain being a fully magnetized single crystal. The remanent magnetization in zero applied field (M_{R}) depends the relative orientation of the domains, which in turn depends on the material properties and domain wall structure. The applied field required to reduce the sample's magnetization to zero is called the coercive field H_{C}, or the coercivity. At sufficiently high applied field all domains become aligned, and the magnetization cannot increase beyond the saturation value (Mₛₐₜ).

The properties of magnetic materials at nano-scale can be substantially different to their bulk properties. This is because nano-scale particles typically comprise only a few, or a single, magnetic domain.

In the presence of an external magnetic field the nanoparticle's magnetization will try to align with the field, as previously described. Whether this is achieved by physical rotation of the particle, or re-alignment the magnetization direction within the particle's crystal lattice is determined by the particle's coercivity (H_{C}), the former behaviour being more likely for high coercivity particles. For the purposes of the invention, physical rotation of the particle is clearly desirable, whilst rotation of magnetization direction within the particle's crystal lattice is undesirable. It is therefore necessary to understand how the coercivity of a nanoparticle can be maximised. FIG. 1b illustrates the difference between the M-H loops of high and low coercivity nanoparticles. A particle with high coercivity is magnetically “hard” whereas one with low coercivity is “soft”.

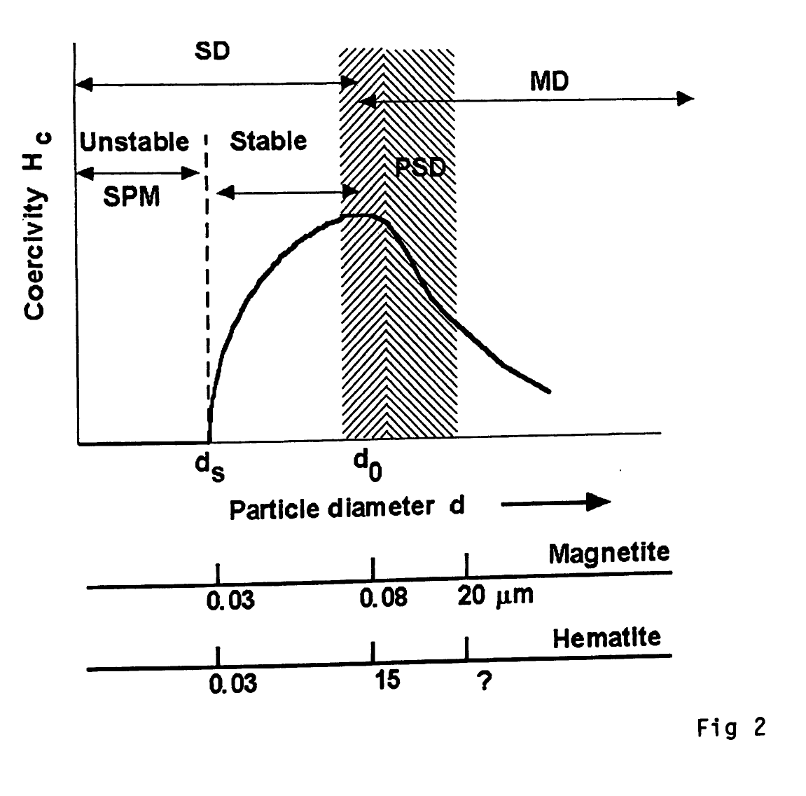

The coercivity depends on particle size, as shown in FIG. 2. Below a critical diameter d₀, the nanoparticle is a single domain (SD), above d₀ it is composed of many domains (MD). The transition region where the particle has a few domains is called the pseudo-single domain range (PSD). The coercivity of the particle peaks near d₀, falling as the particle size and number of domains increases, and also as the size of a single domain decreases. Below a second critical diameter ds the coercivity is effectively nil and the particle displays superparamagnetic behaviour.

Furthermore, a single domain nanoparticle has remanent magnetization equal to the saturation magnetization of a bulk sample, (ie: M_{R}≈Mₛₐₜ), as shown in FIG. 1b. Multi-domain particles have lower remanent magnetization (FIG. 1a).

The variation of coercivity with particle size can be related to material properties as follows. The lower critical size, d_{S}, is controlled by the Neel relaxation time, τ_{N}. For a single domain this is:

$${τ}_{N} = \frac{1}{{f}_{0} \cdot {ⅇ}^{\frac{- {K}_{U} \cdot V}{{k}_{B} \cdot T}}}$$

where k_{B} is Boltzmann's constant, T is absolute temperature, V is particle volume, K_{U} is the magneto-crystalline anisotropy (an intrinsic property of the material) and f₀ is the Neel frequency (10⁹ Hz). The Neel time constant determines how quickly the magnetization can rotate within the crystal lattice in response to a change of applied field; it is determined by the competing effects of thermal energy (k_{B}T) and magneto-crystalline energy (K_{U}V). As V∝d³ it will be appreciated from the equation above that τ_{N} is extremely sensitive to particle size. At constant temperature there exists a critical particle diameter ds for which τ_{N}˜1s:

$${d}_{S} = 3.4 \cdot {( \frac{{k}_{B} \cdot T}{{K}_{U}} )}^{\frac{1}{3}} \qquad ( 2 )$$

For particles smaller than d_{S}, thermal energy dominates, τ_{N} is very short and the nanoparticle's magnetization can rotate freely within the crystal lattice. For single domain particles with diameter greater than d_{S} the relaxation time is very long and the magnetization is effectively locked to the lattice.

The upper critical size d₀ occurs when the energy cost of creating a domain wall is balanced by a net overall reduction in stored magneto-static energy. The magneto-static energy per unit volume is given by:

$${e}_{mag} = \frac{{μ}_{0}}{2} \cdot D \cdot {M}_{sat}^{2}$$

where D is a shape demagnetizing factor. The domain wall energy per unit area is given by:
e_{wall}=4·\sqrt{A≠K_{U}}

where A is the exchange stiffness, of the order 10⁻¹² J/m for ferromagnetic materials.

Hence d₀ may be determined by equating the energy of a single domain particle with that of a two domain particle having the same volume. Assuming the particle is a sphere (and thus D˜⅓):

$${e}_{mag} \cdot [ \frac{4}{3} \cdot π \cdot {( \frac{{d}_{0}}{2} )}^{3} ] = {e}_{mag} \cdot \frac{[ \frac{4}{3} \cdot π \cdot {( \frac{{d}_{0}}{2} )}^{3} ]}{2} + {e}_{wall} \cdot [ π \cdot {( \frac{{d}_{0}}{2} )}^{2} ]$$

which when simplified results in an expression for d₀:

$${d}_{0} = 72 \cdot \frac{\sqrt{A \cdot {K}_{U}}}{{μ}_{0} \cdot {M}_{sat}^{2}} \qquad ( 3 )$$

For sizes above d₀ the particle may re-align its net magnetization in response to a change in applied magnetic field by domain wall movement. Hence coercivity falls as the number of domains increases.

Particles with diameter in the range d_{S}<d<d₀ are referred to as “blocked” or “locked” single domain particles.

The peak coercivity occurs near d₀ and is given approximately by:

$${H}_{C\_peak} = \frac{2 \cdot {K}_{U}}{{μ}_{0} \cdot {M}_{sat}} \qquad ( 4 )$$

For the purposes of the invention it is desirable that the particle is either in the locked single domain range or in the lower part of the multi-domain range (d<˜2d₀), when coercivity is maximised.

Table 1 lists the material properties K_{U} and Mₛₐₜ of various magnetic materials. FIG. 1c shows M-H loops approximately to scale for nanoparticles made from these materials.

TABLE 1
Material properties of some magnetic materials

| | K_{U} (kJ/m³) | Mₛₐₜ (kA/m) |
|---|---|---|
| Magnetite | 15 | 470 |
| Hexaferrite | 100 | 240 |
| SmCo | 10000 | 800 |

Table 2 shows the parameters d_{S}, d₀, and H_{C}_{_{—}}ₚₑₐₖ for the same materials, as calculated from equations (2), (3) and (4).

TABLE 2
Parameters dₛ, d₀, and H_{C}_{_{—}}ₚₑₐₖ for nanoparticles
made from various magnetic materials.

| | dₛ (nm) | d₀ (nm) | H_{C}_{_{—}}ₚₑₐₖ (kA/m) |
|---|---|---|---|
| Magnetite | 22 | 32 | 51 |
| Hexaferrite | 11.7 | 315 | 664 |
| SmCo | 2.5 | 283 | 19900 |

If the applied field is less than the particle's coercive field (H_{applied}<H_{C}), the magnetization will remain effectively locked to the crystal lattice, and the particle will experience a torque acting to physically re-orient it with the applied field direction. However, if the applied field is greater than the particle's coercive field (H_{applied}>H_{C}), the magnetization will rotate within the crystal lattice to align with the applied field direction.

A key aspect of the invention is therefore to provide particles in which the magnetization is locked to the crystal lattice, or “stabilised”, for an applied magnetic field which is sufficiently strong to induce cell killing torque, but much lower than that used in prior art.

This is achieved by one of two alternative methods. The first method suits magnetically “hard” particles, made from materials having inherently high magnetocrystalline anisotropy (K_{U}>10⁵), for example rare-earth alloys (eg: Sm₂CO₁₇), or hexaferrites (eg: BaFe₁₂O₁₉), or tetragonal FePtCr or CoPtCr alloys (K_{U}˜10⁸). The second method suits magnetically “soft” particles, made from materials having inherently low magneto-crystalline anisotropy (K_{U}<10⁵), such as ferrous and ferric oxide (Fe₂O₃ and Fe₃O₄, ie: magnetite).

The two methods are illustrated here by way of numerical examples, assuming the cell killing threshold torque is 100 pN and the particle is a 100 nm diameter sphere (for which M_{eff}˜(⅔)Mₛₐₜ). Considering first hard particles, equation (1) gives the required flux density as 0.036 T for SmCo and 0.12 T for hexaferrite. Converting to field intensity (using H=B/μ₀, where μ₀ is the permeability of free space) the required field is 28 kA/m for SmCo and 95 kA/m for hexaferrite. Table 2 shows that a 100 nm diameter particle made from either material is inside the locked single domain size range. The coercivity H_{C} can be estimated by extrapolation assuming a parabolic fit in the locked SD range, giving about 11000 kA/m for SmCo and 330 kA/m for hexaferrite. So H_{applied}<H_{C} in both cases and the magnetization is locked to the crystal lattice, as required. The key requirement in the first method is to use materials with intrinsically high magneto-crystalline anisotropy (K_{U}>10⁵ J/m³).

Now turning to soft particles, applying equation (1) with M_{eff}·(⅓)Mₛₐₜ (allowing 33% reduction in magnetization due to self de-magnetization and a further, conservative, 33% drop for multi-domain effects) gives a required flux density for a 100 nm magnetite sphere as 0.12 T (˜100 kA/m). Table 2 shows that a 100 nm magnetite particle is well into the multi-domain range, so the particle's coercivity will be significantly lower than the peak of 51 kA/m, probably by a factor of 3 or more. Hence H_{applied}>H_{C} and the magnetization will start to rotate within the lattice at an applied field much less than that required to induce cell killing torque. If the particle diameter is reduced to 32 nm so that the coercivity is maximised at ˜51 kA/m, the required field rises to 950 kA/m (because the particle's volume is now much lower). This too is much greater than the peak coercivity. In fact the magnetization cannot be stabilised for any particle size and the threshold torque cannot be reached. At face value this problem appears to preclude the use of materials with low magneto-crystalline anisotropy.

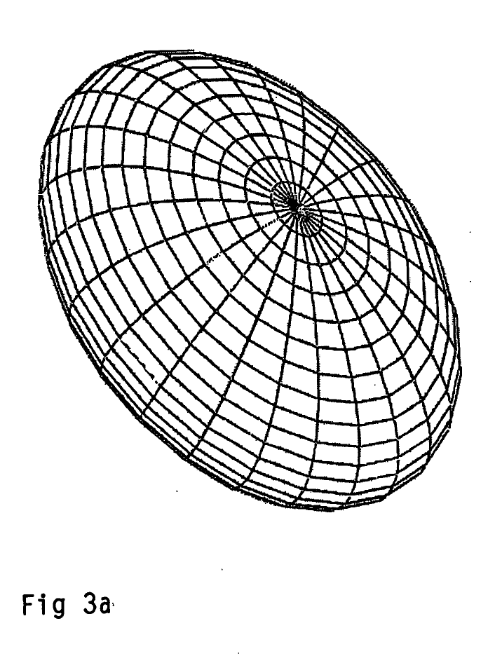

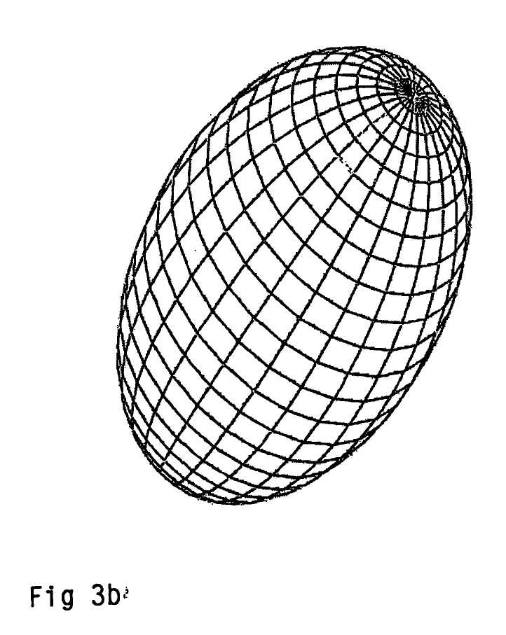

However, it has been found that the magnetization can be effectively locked to the crystal lattice if the magnetic nanoparticles are substantially non-spherical in shape. In other words, shape anisotropy is used as a substitute magneto-crystalline anisotropy. In a particularly preferred embodiment, the soft particles are in the approximate form of either an oblate spheroid with an aspect ratio of approximately 1:2.5 (see FIG. 3a), or a prolate spheroid with an aspect ratio of approximately 1.6:1 (FIG. 3b).

The peak force couple for non-spherical soft particles will occur when the angle between the particle's long axis and the applied field is 45°. The flux density required to generate a maximum force couple F_{C} on a soft spheroid nanoparticle having major axis d, is given by
B=αF_{C}/M_{eff}d² (5)
where the shape factor α=24.4 for the optimum oblate spheroid and 55.6 or the optimum prolate spheroid. Therefore the oblate spheroid (or “disk”) generates over twice as much force as a prolate spheroid (“rod”) with the same maximum dimension. However, the rod has one long axis and two short, whilst the disk has two long, and one short. The orientation of the nanoparticles within the target tissue will be random, so the rod-shaped particle is more likely to find itself at a significant angle to the applied magnetic field than the disk-shaped particle. They are both therefore considered equally preferred shapes.

We can calculate the range of magnetic flux density needed to apply a 100 pN force couple to a 100 nm nanoparticle using equation (1) for materials with high magneto-crystalline anisotropy and equation (5) for particles with low magneto-crystalline anisotropy. The results are contained in Table 3.

TABLE 3
Flux density required to generate 100pN
force couple in 100 nm particles.

| Particle with max dimension of 100 nm | B for 100pN couple (T) |
|---|---|
| Magnetite (optimum disk shape) | 0.52 |
| Magnetite (optimum rod shape) | 1.2 |
| Hexaferrite (sphere) | 0.12 |
| SmCo (sphere) | 0.036 |

Therefore it is clear that magnetically hard particles require a much lower “activation” field strength (ie: field needed to generate threshold torque) compared to magnetically soft particles.

Biological tissue of living organisms can be expected to contain some naturally occurring magnetic material, such as cell magnetosomes (nano-scale magnetite particles occurring in some rare cell types), and iron or iron oxide particulate contamination accumulated over time from the environment. It is clearly desirable that the magnetic therapy does not induce cell damaging torque in these particles. As no control can be exercised over the composition, location or size of the contaminant or natural particles, the only recourse is to minimise the applied field.

Being predominantly iron and iron-oxide the contaminant particles will behave as magnetically soft particles. If the targeted nanoparticles particles are magnetically hard they can be “activated” by a much weaker field than that required to activate the contaminant or naturally occurring particles. In addition to the advantage of lower cost magnet hardware, this is further reason for preferring to use magnetically hard particles or soft particles of the preferred shape.

The particles may be prepared with a coating of a bio-compatible material, which is biologically inert. Examples include polyethylene glycol, ethyleneglycol copolymers, dextrin, polymers and copolymers of hydroxyalkyl(meth)acrylamide, for instance, hydroxypropylmethacrylamide, and copolymers of styrene and maleic anhydride. Additional compounds include polyglutaric acid, carbohydrates and naturally occurring proteins such as albumin. The bonding can be either covalent or non-covalent. The coating will usually be applied to the particles prior to use in the method, however, it is envisaged that particles may attain a coating on administration, e.g. a coating of serum albumin. The preparation of coated particles is known in the art. For example, U.S. Pat. No. 6,048,515, U.S. Pat. No. 5,460,831 and U.S. Pat. No. 5,427,767 all describe coated particles.

The particles may be localised at a target site using any convenient means, including the use of a targeting moiety. The targeting moiety may be any suitable molecule that permits selective targeting to the target site. Examples of suitable targeting moieties include antibodies and receptor ligands, e.g. hormones. Alternatively, localisation may be carried out using the magnetic properties of the particles, with a magnetic field directing the particles to the correct site of action.

In recent years, there has been a great deal of investigation into the use of polymers in the selective targeting of a tumour site. High molecular weight polymers achieve site-specific passive capture through the enhanced permeability and retention (EPR) effect. The EPR effect results from enhanced permeability of macromolecules or small particles within the tumour neovasculature, due to the leakiness of its discontinuous endothelium. In addition to the tumour angiogenesis (hypervasculature) and irregular and incompleteness of vascular networks, the attendant lack of lymphatic drainage promotes accumulation of macromolecules that extravasate. This effect is observed in many solid tumours for macromolecular agents and lipids.

Suitable polymers with which have been used to target tumours by the EPR effect, include polyethylene glycol, ethyleneglycol copolymers, dextrin, polymers and copolymers of hydroxyalkyl(meth)acrylamide, for instance, hydroxypropylmethacrylamide, and copolymers of styrene and maleic anhydride. These may all be used in the present invention.

Improved targeting of the polymers may be achieved by attaching additional ligands to the polymers, e.g. attaching galactose ligands for selective targeting the liver. Antibodies may also be used in conjunction with the polymer to target the tumour site.

Antibodies are the preferred targeting moiety, and there are many examples known in the art of antibodies that have affinity for antigens expressed by aberrant biological structures, e.g. by tumour cells. For example, antibodies raised against the carcinoembryonic antigen (CEA) are known. A review of antibodies raised against human tumour antigens is found in Lloyd, Basic and Clinical Tumour Immunology (Herberman, Ed.), 1983: 159-214.

Any suitable antibody or fragment may be used in the present invention. Suitable antibodies include recombinant antibodies, antibodies from natural sources, e.g. human, molecules such as Fab fragments, Fab′ fragments, F(ab′)₂ fragments, Fv and SCFv. Antibodies that permit internalisation into a cell are a preferred embodiment of the invention. These so-called “internalising antibodies” are known to those skilled in the art. The antibodies will usually have an affinity for a target of at least 10⁻⁷M, preferably at least 10⁻⁹M and most preferably at least 10⁻¹¹M. The preparation of antibodies suitable for use in the invention will be apparent to those skilled in the art.

Examples of techniques used to prepare antibodies can be found in Molecular Cloning: A Laboratory Manual, 2nd Edition, Sambrook et al., Cold Spring Harbour, N.Y. (1989). Additional teachings can be found in U.S. Pat. No. 4,816,397, U.S. Pat. No. 4,816,567, GB 2188638 and GB 2209757.

In the preferred embodiment, the antibodies are bound to the magnetic particles. However, it is also envisaged that the antibody may be localised at the target site independently of the magnetic particles, with a suitable antigen being attached to the magnetic particles for targeting the antibody.

There may be more than one targeting moiety attached to each particle.

The targeting moiety may be bound to the magnetic particle through one or more linkages, which may be covalent or non-covalent. The targeting moiety may be attached to the magnetic particle using a cleavable linker molecule. Examples of suitable cleavable linkers are known in the art.

Attachment of the targeting moiety may be achieved using any suitable linker molecule that attaches directly to the magnetic particle or attaches to a coating on the magnetic particle. Suitable linker groups will be apparent to those skilled in the art and include: polyethylene glycol (PEG), polyols including polysaccharides and polycarboxylates. Attachment of antibodies to nano-particles is disclosed in U.S. Pat. No. 6,514,481.

In one embodiment, the magnetic particle (or its coating) is attached to the targeting moiety via a multidentate ligand which results in improved stability of the binding interaction. Multidentate ligands comprise linked multiple sites of attachment between the particle and the targeting moiety. The targeting moiety is therefore attached to the particle by more than one linkage, resulting in improved stability.

The magnetic particles may be used to target and disrupt any suitable material. Disruption may be carried out for a therapeutic or diagnostic purpose and targeting may be carried out in vitro or in vivo. Preferably, the material is a biological material, e.g. an aberrant cellular or tissue structure, more preferably a mammalian cellular or tissue structure. For example, the magnetic particles of the invention are particularly suitable for the treatment of a tumour which can be targeted due to the expression of specific tumour-associated antigens. The particles will localise at the surface of the cell membrane or may be internalised, prior to the application of the magnetic field. Once activated by the magnetic field, the particles disrupt internal or external cellular structures, resulting in cell death. The particles are then removed from the body by normal excretory mechanisms such as macrophage action.

Tumours which may be treated using the present invention will be apparent to the skilled person. The tumours may be solid or liquid tumours (e.g. leukaemia), and may be macro or micro tumours.

The material may also be a cell comprising an infectious agent. The material may also be a multicellular or unicellular microorganism, e.g. a prokaryotic or eukaryotic microorganism. The material may also be viral or fungal.

In a separate embodiment, the particles may be used to disrupt or dislodge unwanted biological material at a target site. An example of this is in the treatment of artherosclerotic plaques. It is possible to localise the magnetic particles at this site of the plaque by denatured or oxidised LDL, and subsequent activation of the particles will reduce the density of the plaque by dislodging the sclerotic material. Antibodies that have affinity to plaques are discussed in U.S. Pat. No. 6,379,699.

The particles may be administered to a patient in a variety of ways. Preferably, the particles are formulated in compositions for parenteral administration, i.e. subcutaneous, intramuscular or intravenous administration. Accordingly, the invention provides compositions for parenteral administration which comprise the particles dispersed in a suitable carrier material, preferably an aqueous carrier material. Suitable carriers are known to those skilled in the art. The compositions will usually be prepared in unit dosage form. Preferably, a unit dose will comprise a maximum of 100 mg/kg of the targeting moiety.

The particles may be administered using transdermal or ballistic delivery devices, eg needleless injection devices. The general principle of ballistic administration is the use of a supersonic wavefront, created by the release of compressed gas, to propel the particles to a target site (see Vain et al., Plant Cell, Tissue and Organ Culture, 1993; 33:237-246). Devices have also been described which deliver medicines using gas pressure, eg U.S. Pat. No. 4,790,824 and PCT/GB94/00753.

The particles may be formulated as “dry” compositions for delivery using ballistic devices, or the particles may be suspended in any pharmaceutically acceptable buffer, diluent or excipient for other suitable routes of administration.

In one embodiment, the particles are delivered to a patient via slow infusion, maintaining an essentially homogeneous dispersion of the particles in an aqueous carrier material.

The present invention is intended for treatment of human patients and for veterinary applications. However, in vitro and ex vivo applications are also envisaged.

A particular advantage of the invention for the treatment of cancer is the potential ability to treat all malignant cells in a patient's body, not just those in the main tumour. This is a benefit of the antibody targeting technology, which targets cells having a particular characteristic in any tissue that is reached by the patient's circulatory system. This confers a distinct advantage over other “targeted” methods that restrict the therapy to a particular anatomical location; for example, the invention described here is less likely to miss metastases.

To take advantage of this, it is preferred that the patient's entire body is subjected to the therapeutic magnetic field. Therefore magnet hardware that is large enough to treat the whole body is preferred, although smaller scale magnets are possible for anatomically localised treatment, including mobile or even hand-held units for low field strength operation. The examples of suitable magnet hardware given here are for whole body units but it is intended that this application covers the option of smaller scale hardware dedicated to particular parts of the anatomy, the basic design principles of which will be apparent to those skilled in the art.

A whole body magnet does not have to treat the entire body simultaneously; it is acceptable to treat a portion at a time. This approach significantly reduces the cost and complexity of the magnet hardware because the working region, over which the magnetic field has the required parameters, need only be large enough to encompass the largest anatomical structure, typically the abdomen. The preferred embodiment therefore creates an approximately spheroid working region in a gap in the magnetic circuit. The patient is passed through the gap, thus exposing the entire body to treatment.

In order to damage the cells, the magnetic nanoparticles must move, and in order to move they must experience a changing force, and hence a changing magnetic field. A range of magnet design options are presented here, covering the range of field strengths required to generate cell damaging forces for a 100 nm particle, as calculated above. In each case, the practical range of possible field slew rates and effective field oscillation frequency is estimated. It will be apparent that these figures will change if the assumptions about particle size or cell damage force threshold change, but the principles of the invention remain valid.

In the simplest embodiment it is possible to simply pulse the magnetic field on and off. When the field is on, particles will attempt to rotate to align with the field. In the absence of an applied field, the particles' orientations will be random, therefore those particles that are already aligned with the field by chance will not experience any torque. It is therefore also desirable to change the direction of the applied field, so that all particles have a good chance of experiencing maximum torque. A rotating field will cause the particles to rotate to follow the field direction, thus maximising chances for cell damage. It is also desirable, but not required, to modulate the field amplitude over time, either continuously or in a pulsed fashion.

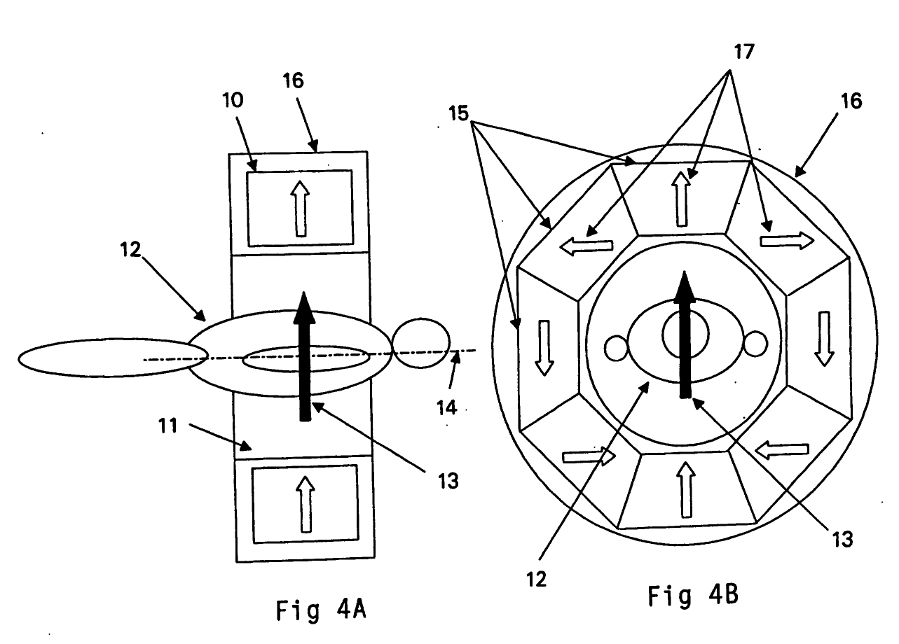

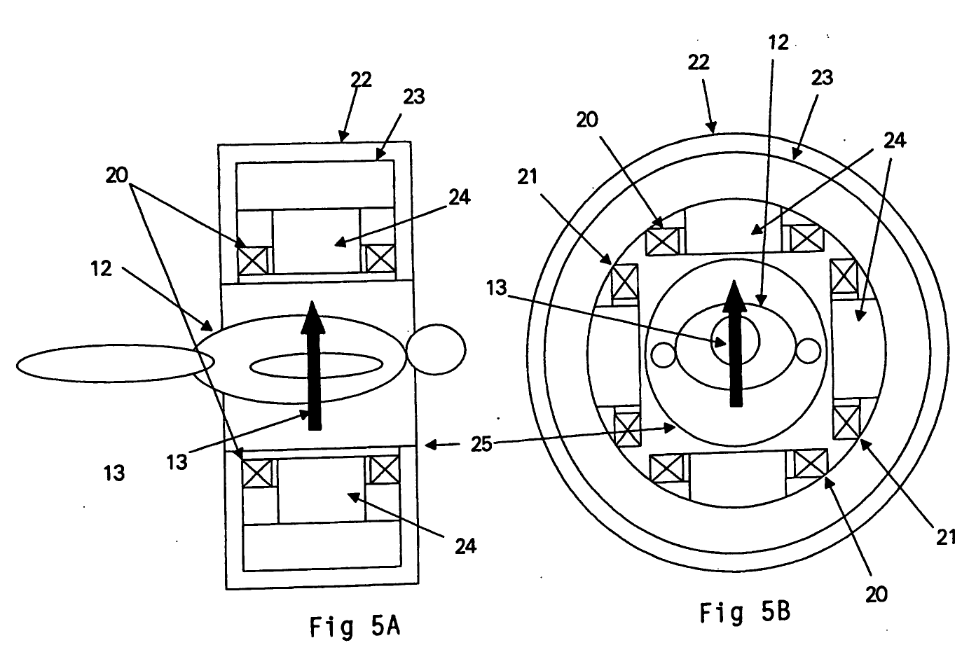

In one embodiment, the field direction or amplitude is varied at a frequency up to 100 Hz, preferably up to 50 Hz and more preferably up to 10 Hz. The change in field direction can be accomplished either by moving (eg: rotating) the patient within a static magnetic field or varying the field applied to the patient. The latter can be achieved either by physically rotating the magnet hardware (FIG. 4) or by modulating currents in static electromagnet coils, preferably mounted on a soft iron yoke (FIG. 5). In either case, the magnetic field direction should be directed perpendicular to the rotation axis, ie: across the working gap. Various hardware options are feasible to generate such a field, each suited to a different mode of operation and field strength.

FIG. 4A is a longitudinal cross-section and FIG. 4B a transverse cross-section of a ring-shaped magnet assembly made from prismatic blocks of rare-earth permanent magnet material 15 having fixed magnetization direction shown by arrows 17 defining a Halbach cylinder assembly 10, as known from prior art. Such a structure generates a fairly uniform magnetic field, directed as indicated by an arrow 13, across the bore 11 of a support structure 16 in which a patient 12 is inserted. The magnetic field direction 13 which can be rotated with respect to the patient by rotating the entire magnet assembly 10 around its axis 14. Field strengths of up to 0.2 T are envisaged with a slew rate of up to or about 3 T/sec, equivalent to physical rotation of the magnet at about 450 rpm, although faster rotation would be possible with suitable mechanical design. The equivalent field oscillation frequency in this example is about 7.5 Hz.

FIGS. 5A and 5B are similar to FIGS. 4A and 4B but illustrating a second example in which a set of coils 20, 21 are located on pole pieces 24 connected by a soft iron yoke 23 within a housing 22 having a bore 25 (22 and 25 may define a cryostat). The electromagnets may be resistive or made from high (or low) temperature superconductor. In the case of resistive coils, water cooling will be needed to remove DC losses. In the case of superconducting coils, cryogenic cooling is needed. Rotation of the field direction 13 is caused by driving current in the coils 20, 21 in quadrature. This is a two phase structure but three or more phases could be implemented by using more electromagnets. In the case of superconducting coils, the cryostat must be designed with a high cooling power to absorb AC losses. For this reason, high temperature superconductors may be preferred, as the cost of providing cryogenic cooling power is inversely proportional to temperature. Magnetic field strengths up to about 0.5 T are possible with a slew rate in the range of 1-5 T/sec, equivalent to a maximum field oscillation frequency of approximately 2.5 Hz.

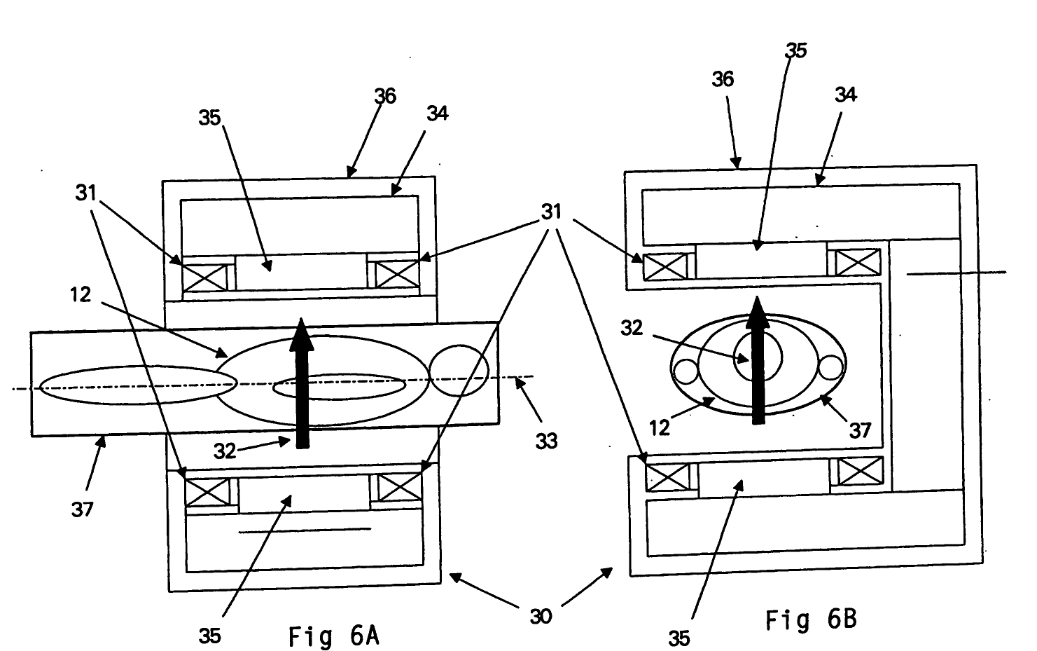

FIG. 6 illustrates a C-shaped magnet 30 (in transverse cross-section in FIG. 6A and longitudinal cross-section in FIG. 6B which is similar to the “open” or C-type whole body magnet conventionally used for magnetic resonance imaging (MRI), with coils 31 formed from low temperature superconducting wire mounted on a pole 35 connected by a support structure 34, which may optionally be a soft iron yoke to guide flux, which generate a magnetic field having a direction indicated by an arrow 32. The patient 12 is mounted on a support 37 and rotated about their long axis 33. The coils will be located within a cryostat 36 in a conventional manner. In this example, a static magnetic field up to about 1 T is envisaged with maximum rotation speed of the patient of about 150 rpm. This corresponds to an effective field rotation frequency of 2.5 Hz.

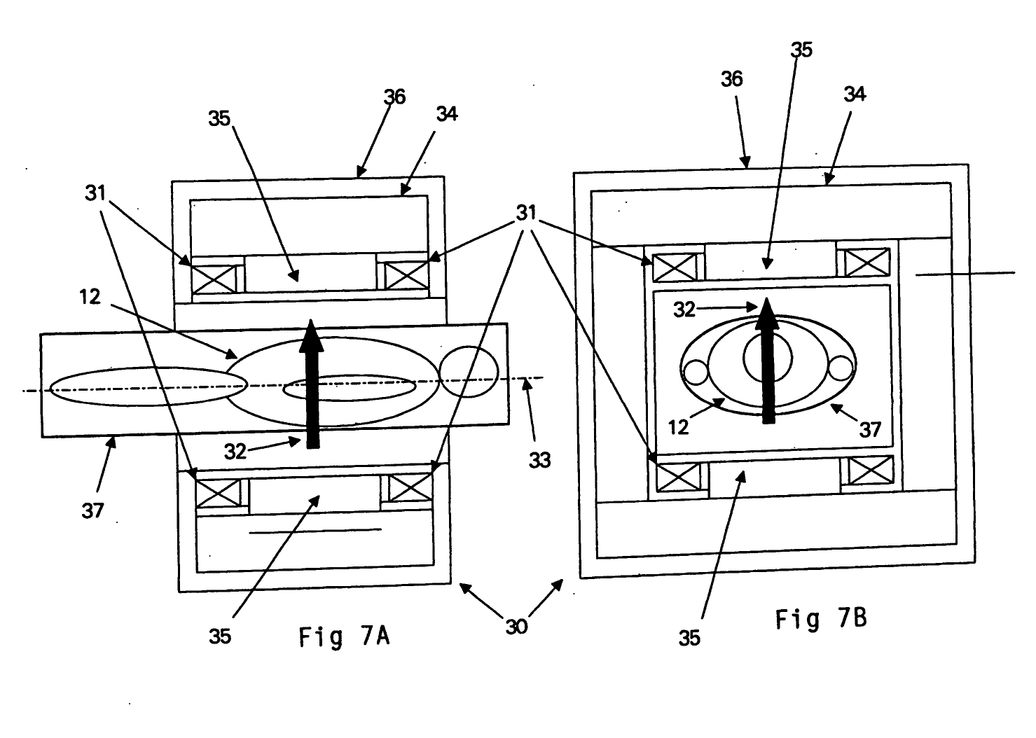

FIG. 7 is an alternative arrangement of FIG. 6, with a second flux return arm, forming a “window-frame” magnet. In all other respects it is similar to the C-magnet.

A further alternative magnet arrangement is based on the example shown in FIGS. 6 and 7. The magnetic field direction 32 in this embodiment is fixed and the patient is not rotated but the field amplitude is pulsed by varying the current sent to the coils, which may be wound from copper wire or high temperature superconducting wire, as required. Between pulses the magnet hardware or patient can be rotated to a new position to offer the maximum chance of applying a torque impulse to all particles, regardless of their orientation. However, in practice the orientation of the particles will be randomised by Brownian motion, so this step may not be required. The Brownian rotational diffusion time constant is given by
τ_{B}=3Vη/k_{B}T
where η is viscosity. For a 100 nm particle this time constant ranges from milliseconds for water (η=0.89 centipoise) to seconds for the more viscous fluids which are likely to be more representative of the intracellular environment (η˜tens of poise). If the time constant for Brownian randomization of particles is short enough it may be acceptable to simply wait for a few seconds before repeating the magnetic field pulse.

In general, some embodiments will share many common features with a magnet for magnetic resonance imaging. However it is important to note that the uniformity of field in the working region required for an MRI magnet is not a requirement for a therapy magnet for use in the current invention. A variation of flux density of 20-30% within the working region is quite acceptable (provided the weakest field is stronger than the threshold for cell damage). In consequence, some magnet embodiments for the present invention may have reduced cost compared to MRI magnets.

As mentioned above, MRI type magnets can be used and in this case, it is feasible that an MR image could be obtained at various stages during the process, and also before and after the process, of the structure in the working volume.

Even if the therapy magnet is not capable of MRI, it may be desirable to use a conventional MRI system to image the patient after administration of the nanoparticles and before magnetic therapy, to ensure correct location of the nanoparticles, which will appear in a suitably constructed MR imaging sequence due to their strong magnetic properties. Such a pre-screening process will also be useful for identifying large ferromagnetic foreign bodies in the patient's body, such as metallic swarf, that would represent a hazard during magnetic therapy.

Although the present invention has been described with emphasis on the disruption of biological material in a therapeutic context, other (non-therapeutic) applications of the disruption method are envisaged. For example, the build up of non-organic materials may be disrupted. In addition, cosmetic applications of the method are also envisaged.

What is claimed is:

1. A method for treating a disorder associated with a cellular or tissue structure or the accumulation of an undesirable biological material in a subject comprising administering to the subject one or more magnetic particles, wherein at least one particle localizes at or within the structure or material, and wherein the treatment is carried out by applying a magnetic field, to induce at least one particle to rotate, to thereby disrupt the structure or material, wherein at least one magnetic particle has intrinsic magnetization, said magnetization being stabilized by inherent magnet crystalline anisotropy and/or by shape anisotropy.

2. The method of claim 1, wherein the structure is a mammalian cell.

3. The method of claim 1, wherein the structure is a tumor.

4. The method of claim 1, wherein at least one particle comprises a targeting moiety.

5. The method of claim 4, wherein the targeting moiety is an antibody.

6. The method of claim 5 wherein the antibody is a cell-internalizing antibody.

7. The method of claim 1, wherein at least one particle comprises a magnetic material with a magneto-crystalline anisotropy of at least 1×10⁵ J/m³.

8. The method of claim 7, wherein the magnetic material is a rare-earth metal alloy or a crystalline hexaferrite.

9. The method of claim 1, wherein at least one particle comprises a coating of a bio-compatible material.

10. The method of claim 7, wherein the magnetic material of at least one particle is of a maximum dimension of from 50 nm to 500 nm.

11. The method of claim 1, wherein at least one particle has a total maximum dimension not exceeding 200 nm.

12. The method of claim 1, wherein one or more particles is a shape selected from the group consisting of: substantially cuboid, substantially oblate spheroid and substantially prolate spheroid.

13. A method for disrupting a material, comprising the steps of:
(i) localizing one or more magnetic particles at or within the material: and
(ii) applying a magnetic field to one magnetic particle, to induce particle rotation and thereby disrupt the material, wherein one magnetic particle has intrinsic magnetization, said magnetization being stabilized by inherent magneto-crystalline anisotropy and/or by shape anisotropy and wherein the applied magnetic field direction and/or amplitude with respect to the material is varied over time.

14. The method of claim 13, wherein the material is a biological material.

15. The method of claim 13, wherein the material is a cellular or tissue structure.

16. The method of claim 13, wherein the material is a mammalian cell.

17. The method of claim 13, wherein the material is a tumor.

18. The method of claim 13, wherein the method is performed in vitro.

19. The method of claim 13, wherein at least one particle comprises a targeting moiety.

20. The method of claim 19, wherein the targeting moiety is an antibody.

21. The method of claim 20, wherein the antibody is a cell-internalizing antibody.

22. The method of claim 13, wherein the at least one particle comprises a magnetic material with a magneto-crystalline anisotropy of at least 1×10⁵ μm³.

23. The method of claim 22, wherein the magnetic material is a rare-earth metal alloy or a crystalline hexaferrite.

24. The method of claim 13, wherein at least one particle comprises a coating of a bio-compatible material.

25. The method of claim 22, wherein the magnetic material of at least one particle is of a maximum dimension of from 50 nm to 500 nm.

26. The method of claim 13, wherein at least one particle has a total maximum dimension not exceeding 200 nm.

27. The method of claim 13, wherein one or more particles is a shape selected from the group consisting of: substantially cuboid, substantially oblate spheroid and substantially prolate spheroid.

28. The method of claim 13, wherein the applied magnetic field has a flux density of from 0.01 to 2 Tesla.

29. The method of claim 13, wherein the magnetic field variation is continuous.

30. The method of claim 13, wherein the variation is discontinuous, the magnetic field being repeatedly applied after re-orienting the material.

31. The method of claim 13, wherein the variation is discontinuous, the magnetic field being repeatedly applied after a predetermined wait period to allow the magnetic axis of at least one particle to take up a random direction as a result of Brownian motion.

32. The method of claim 13, wherein the variation is achieved by suitably controlling an external magnetic field generator.

33. The method of claim 13, wherein the field direction is varied at a frequency up to 100 Hz.

34. The method of claim 13, wherein the variation is achieved by moving the material.

35. The method of claim 13, wherein the material is rotated at a frequency up to 10 Hz.

36. The method of claim 13, further comprising obtaining a magnetic resonance image of at least one particle prior to causing movement of at least one particle.

37. An apparatus for disrupting a material, the apparatus comprising a magnetic field generator for generating a magnetic field in a working volume; one or more magnetic particles localized at or in the material in the working volume, wherein at least one magnetic particle has intrinsic magnetization, said magnetization being stabilized by inherent magneto-crystalline anisotropy and/or by shape anisotropy; and a control system for causing a change in the magnetic field in the working volume with respect to the material so as to rotate the magnetic particle.

38. The apparatus of claim 37, wherein the control system causes a relative movement between the magnetic field direction and the material.

39. The apparatus of claim 38, wherein the control system causes a relative rotation between the magnetic field direction and the working volume.

40. The apparatus of claim 37, wherein the control system is adapted to cause the magnetic field vector in the working volume to change with respect to the material in amplitude or direction or both.

41. The apparatus of claim 40, wherein the control system is adapted to cause the magnetic field generator to change relative to the material.

42. The apparatus of claim 37, wherein the control system is adapted to cause the magnetic field generator to pulse the amplitude of the magnetic field in the working volume.

43. The apparatus of claim 37, wherein the working volume is located externally of the magnetic field generator.

44. The apparatus of claim 37, wherein the magnetic field generator comprises one or more electromagnets.

45. The apparatus of claim 44, wherein at least one electromagnet is fabricated from a high temperature superconductor.

46. The apparatus of claim 37, wherein the magnetic field generated by the magnetic field generator has a field strength in the range 0.01 to 2 Tesla.

47. The apparatus of claim 37, wherein at least one particle comprises a targeting moiety.

48. The apparatus of claim 47, wherein the targeting moiety is an antibody.

49. The apparatus of claim 48, wherein the antibody is a cell-internalizing antibody.

50. The apparatus of claim 37, wherein at least one particle comprises a magnetic material with a magneto-crystalline anisotropy of at least 1×10⁵ J/m³.

51. The apparatus of claim 50, wherein the magnetic material is a rare-earth metal alloy or a crystalline hexaferrite.

52. The apparatus of claim 37, wherein at least one particle comprises a coating of a bio-compatible material.

53. The apparatus of claim 50, wherein the magnetic material of at least one particle is of a maximum dimension of from 50 nm to 500 nm.

54. The apparatus of claim 37, wherein at least one particle has a total maximum dimension not exceeding 200 nm.

55. The apparatus of claim 37, wherein one or more particles is a shape selected from the group consisting of: substantially cuboid, substantially oblate spheroid and substantially prolate spheroid.

56. A composition comprising a plurality of the magnetic particles of claim 4, and optionally, a pharmaceutically acceptable buffer, excipient or diluent.

57. A composition comprising a plurality of the magnetic particles of claim 5, and optionally, a pharmaceutically acceptable buffer, excipient or diluent.

58. A composition comprising a plurality of the magnetic particles of claim 6, and optionally, a pharmaceutically acceptable buffer, excipient or diluent.

59. A composition comprising a plurality of the magnetic particles of claim 7, and optionally, a pharmaceutically acceptable buffer, excipient or diluent.

60. A composition comprising a plurality of the magnetic particles of claim 8, and optionally, a pharmaceutically acceptable buffer, excipient or diluent.

61. A composition comprising a plurality of the magnetic particles of claim 9, and optionally, a pharmaceutically acceptable buffer, excipient or diluent.

62. A composition comprising a plurality of the magnetic particles of claim 10, and optionally, a pharmaceutically acceptable buffer, excipient or diluent.

63. A composition comprising a plurality of the magnetic particles of claim 11, and optionally, a pharmaceutically acceptable buffer, excipient or diluent.

64. A composition comprising a plurality of the magnetic particles of claim 12, and optionally, a pharmaceutically acceptable buffer, excipient or diluent.

65. A composition according to claim 40, for therapeutic use.

66. The method of claim 1, wherein the particle is to be rotated to exert a force of at least 100 pN.

67. The method of claim 13, wherein the particle is to be rotated to exert a force of at least 100 pN.
